(19) United States
(12) Patent Application Publication
(10) Pub. No.: US 2025/0318557 A1
(43) Pub. Date: Oct. 16, 2025

(54) METHOD FOR SUPPRESSING OBESITY OR REDUCING BODY FAT BY ADMINISTERING AN ORAL COMPOSITION CONTAINING TECTORIGENINS

(71) Applicant: TOYO SHINYAKU CO., LTD., Fukuoka (JP)

(72) Inventors: Hiroshi TOMOZAWA, Saga (JP); Hideki UEDA, Saga (JP); Aki MIYAMOTO, Saga (JP); Tomoyasu KAMIYA, Saga (JP)

(73) Assignee: TOYO SHINYAKU CO., LTD., Fukuoka (JP)

(21) Appl. No.: 19/247,623

(22) Filed: Jun. 24, 2025

Related U.S. Application Data
(63) Continuation of application No. 17/908,354, filed on Aug. 31, 2022.

(30) Foreign Application Priority Data
Mar. 31, 2020 (JP) 2020-063897

(51) Int. Cl. A23L 27/20; A23L 27/21; A23L 27/23; A23L 27/30

(52) U.S. Cl. CPC A23L 27/2052; A23L 27/2026; A23L 27/21; A23L 27/23; A23L 27/33

(57) ABSTRACT

A method for suppressing obesity or for reducing body fat that includes administering to a human an oral composition containing tectorigenins, and one or more compounds selected from anthocyanins, quercetins, glutamic acid, histidine, methionine, phenylalanine, tryptophan, cysteine, glycine, nucleotides, and inulins. The oral composition may be in the form of a powder, a granule, a tablet, or a capsule.

### CROSS-REFERENCE TO RELATED APPLICATIONS

This is a Continuation of U.S. application Ser. No. 17/908,354, which is the U.S. National Stage entry of PCT/JP2020/036767, filed Sep. 29, 2020, which claims priority to JP Application No. 2020-063897, filed Mar. 31, 2020. The disclosure of each of these applications is herein incorporated by reference in its entirety.

### TECHNICAL FIELD

The present invention relates to an oral composition comprising tectorigenins and a specific compound.

### BACKGROUND ART

Flavonoids are a general term for compounds such as flavones and isoflavones, and are known to have physiological activities that are beneficial to people. In recent years, due to growing awareness of health care, active intake of flavonoids has been recommended.

Tectorigenins are known as one of the flavonoids that have beneficial physiological activities. Tectorigenins are compounds contained in plants of the family Iridaceae, or the like. As for the physiological activities that tectorigenins have, for example, an action of ameliorating malfunction of the urogenital system associated with sex hormones (Patent Literature 1), an action of activating sirtuins (Patent Literature 2), and others are known. Since tectorigenins are flavonoids that are beneficial for maintaining or improving health, there is a need to develop oral compositions comprising tectorigenins.

### CITATION LIST

### Patent Literature

Patent Literature 1: Japanese Unexamined Patent Application Publication (Translation of PCT Application) No. 2005-500999
Patent Literature 2: Japanese Laid Open Patent Publication No. 2006-298876

### SUMMARY OF INVENTION

### Technical Problem

However, tectorigenins have a unique taste, making them difficult to be taken orally on a continuous basis. In addition, since the method for suppressing decomposition of tectorigenins and maintaining them stably has not been adequately studied as well, there is a concern that the amount of tectorigenins comprised in oral compositions may decrease during storage. Therefore, there is a need to ameliorate the taste of oral compositions comprising tectorigenins and to ameliorate the stability of such oral compositions.

The present invention has been made, as for oral compositions comprising tectorigenins, for the purpose of ameliorating the taste of oral compositions comprising tectorigenins and ameliorating the stability of such oral compositions.

### Solution to Problem

As a result of diligent investigations in order to solve the above problems, the present inventors have found that the combination of tectorigenins and a specific compound can ameliorate the taste or stability of oral compositions comprising tectorigenins, thereby completed the present invention.

The present inventors have also found that the combination of tectorigenins and a specific compound demonstrates an excellent anti-obesity effect, thereby leading to the present invention.

Specifically, the present invention is as follows.

Summary of the present invention is as follow.
<1> An oral composition comprising tectorigenins, and one or more compounds selected from the group consisting of (A) to (D):
   (A) one or more polyphenols selected from anthocyanins and quercetins;
   (B) one or more amino acids selected from glutamic acid, histidine, methionine, phenylalanine, tryptophan, cysteine, and glycine;
   (C) nucleotides; and
   (D) inulins.
<2> An oral composition comprising tectorigenins, and two or more compounds selected from the group consisting of (A) to (D):
   (A) one or more polyphenols selected from anthocyanins and quercetins;
   (B) one or more amino acids selected from glutamic acid, histidine, methionine, phenylalanine, tryptophan, cysteine, and glycine;
   (C) nucleotides; and
   (D) inulins.
<3> An oral composition comprising tectorigenins, one or more compounds selected from (A), and one or more compounds selected from (B) to (D).
   (A) one or more polyphenols selected from anthocyanins and quercetins;
   (B) one or more amino acids selected from glutamic acid, histidine, methionine, phenylalanine, tryptophan, cysteine, and glycine;
   (C) nucleotides; and
   (D) inulins.
<4> An oral composition comprising tectorigenins, one or more compounds selected from (B), and one or more compounds selected from (C) or (D).
   (B) one or more amino acids selected from glutamic acid, histidine, methionine, phenylalanine, tryptophan, cysteine, and glycine;
   (C) nucleotides; and
   (D) inulins.
<5> An oral composition comprising tectorigenins, one or more compounds selected from (B), and (C).
   (B) one or more amino acids selected from glutamic acid, histidine, methionine, phenylalanine, tryptophan, cysteine, and glycine;
   (C) nucleotides.
<6> The oral composition according to any one of <1> to <5>, wherein the (C) nucleotides are any one or more of inosinic acid, deoxyribonucleic acid (DNA), or ribonucleic acid (RNA).
<7> An anti-obesity composition comprising tectorigenins, and one or more compounds selected from the group consisting of (A) to (D):
   (A) one or more polyphenols selected from anthocyanins and quercetins;
   (B) one or more amino acids selected from glutamic acid, histidine, methionine, phenylalanine, tryptophan, cysteine, and glycine;
   (C) nucleotides; and
   (D) inulins.
<8> The anti-obesity composition according to <7>, wherein the (C) nucleotides are any one or more of inosinic acid, deoxyribonucleic acid (DNA), or ribonucleic acid (RNA).
<9> A composition for reducing body fat comprising tectorigenins, and one or more compounds selected from the group consisting of (A) to (D):
   (A) one or more polyphenols selected from anthocyanins and quercetins;
   (B) one or more amino acids selected from glutamic acid, histidine, methionine, phenylalanine, tryptophan, cysteine, and glycine;
   (C) nucleotides; and
   (D) inulins.
<10> The composition for reducing body fat r according to <9>, wherein the (C) nucleotides are any one or more of inosinic acid, deoxyribonucleic acid (DNA), or ribonucleic acid (RNA).

### Advantageous Effects of Invention

According to the present invention, an oral composition which taste, such as deliciousness, sweetness, and umami taste, has been ameliorated by comprising tectorigenins and a specific compound can be provided. In addition, according to the present invention, an oral composition with ameliorated storage stability of tectorigenins can be provided. Also, according to the present invention, an oral composition having an excellent anti-obesity effect can be provided.

### DESCRIPTION OF EMBODIMENTS

Hereinafter, the present invention will be described in detail.

### Tectorigenins

In the present invention, tectorigenins mean tectorigenin, tectoridin, and tectorigenin 7-O-xylosylglucoside (TGXG), or mixtures thereof. The tectorigenins used in the present invention may be one or more selected from tectorigenin, tectoridin, and TGXG, but two or more selected from tectorigenin, tectoridin, and TGXG are more preferred, and a mixture of tectorigenin, tectoridin, and TGXG is particularly preferred. The tectorigenins may be those synthesized or those comprised in plants.

The amount of tectorigenins in the composition of the present invention can be measured by the HPLC method. For example, the measurement can be performed by using YMC-Pack ODS AM12S05-2546WT (φ4.6×250 mm) manufactured by YMC Co., Ltd., using an acetonitrile/water/acetic acid mixed solution (mobile phase A volume ratio=15:85:0.1, mobile phase B volume ratio=35:65:0.1) as the liquid medium for the mobile phase, and setting the column temperature and flow rate to 35° C. and 1.0 ml/min, respectively. The gradient conditions can be as follows.

TABLE 1

| Time | A liquid | B liquid |
|---|---|---|
| (min) | (%) | (%) |
| 0 | 100 | 0 |
| 50 | 0 | 100 |
| 55 | 0 | 100 |
| 57 | 100 | 0 |
| 70 | 100 | 0 |

In the oral composition of the present invention, there is no particular limitation on the content of the tectorigenins, but from the viewpoint of taste improvement, stability improvement, and anti-obesity effect, it is preferably 0.001% by weight or more, more preferably 0.005% by weight or more, still more preferably 0.008% by weight or more, and particularly preferably 0.01% by weight or more, with respect to the entire amount of the oral composition of the present invention. The upper limit of the content is not particularly limited, but from the viewpoint of taste improvement, stability improvement, and anti-obesity effect, it is preferably 20% by weight or less, more preferably 10% by weight or less, still more preferably 8% by weight or less, and particularly preferably 5% by weight or less. Note that the content in a case where the oral composition is in the form of a tea bag means the content in the extract liquid. The content of the tectorigenins in this case refers to the total amount of tectorigenin, tectoridin, and TGXG.

In the oral composition of the present invention, there is no particular limitation on the intake amount of the tectorigenins, but from the viewpoint of taste amelioration, stability improvement, and anti-obesity effect, the intake amount of the tectorigenins per day is preferably 1 mg/day or more, more preferably 5 mg/day or more, still more preferably 10 mg/day or more, and particularly preferably 20 mg/day or more. The upper limit of the intake amount is not particularly limited, but from the viewpoint of taste amelioration, stability improvement, and anti-obesity effect, it is preferably 2 g/day or less, more preferably 1 g/day or less, still more preferably 0.8 g/day or less, and particularly preferably 0.5 g/day or less. The oral composition of the present invention can be stored in a single container so that the daily intake amount is the aforementioned intake amount, or it can be divided into and stored in several, for example, two to three containers as a daily dose. The intake amount of the tectorigenins in this case refers to the total amount of tectorigenin, tectoridin, and TGXG.

### Specific Compounds

In the present invention, at least one or more compounds selected from the group consisting of (A) to (D) (abbreviated as “specific compounds”) are comprised together with the tectorigenins. Hereinafter, the specific compounds will be described in detail.

The specific compound used in the composition of the present invention is at least one or more compounds selected from the group consisting of (A) one or more polyphenols selected from anthocyanins and quercetins, (B) one or more amino acids selected from glutamic acid, histidine, methionine, phenylalanine, tryptophan, cysteine, and glycine, (C) nucleotides, and (D) inulins.

The compounds (A) to (D) comprised together with the tectorigenins may be used as a single type (for example, one type of the compounds of (A)), or may be used in combination of two or more types (for example, two types from the compounds of (A), or a compound of (A) and a compound of (B)). In the present invention, from the viewpoint of taste amelioration, stability improvement, and anti-obesity effect, it is preferable to comprise two or more compounds selected from (A) to (D), it is more preferable to comprise one or more compounds selected from (A) and one or more compounds selected from (B) to (D), or to comprise one or more compounds selected from (B) and one or more compounds selected from (C) and (D), and it is particularly preferable to comprise one or more compounds selected from (A) and one or more compounds selected from (B) and (C), or to comprise one or more compounds selected from (B) and one or more compounds selected from (C).

### A. Polyphenols

The polyphenols used in the present invention are one or more selected from anthocyanins and quercetins. Anthocyanins are glycosides of anthocyanidins. Quercetin is one of flavonols. The quercetins in the present invention are a concept that encompasses glycosides. The anthocyanins or quercetins used in the present invention may be those synthesized or those comprised in plants. When the polyphenols used in the present invention are derived from plants, it is preferable that they be derived from plants other than those comprising tectorigenins.

### B. Amino Acids

The amino acids used in the present invention are one or more selected from glutamic acid, histidine, methionine, phenylalanine, tryptophan, cysteine, and glycine. The amino acids in the present invention mean free amino acids and do not include amino acids constituting proteins. The amino acids used in the present invention may be those synthesized or those comprised in plants or animals. When the amino acids used in the present invention are derived from plants, it is preferable that they be derived from plants other than those comprising tectorigenins.

### C. Nucleotides

The nucleotides in the present invention are a concept that encompasses nucleotides, which are phosphate esters of nucleosides, and polynucleotides (nucleic acids), which are conjugates of nucleotides. As the phosphate esters of nucleosides, for example, taste-presenting nucleotides such as inosinic acid (IMP) and guanylic acid (GMP), ribonucleotides such as adenylic acid (AMP), adenosine diphosphate (ADP), and adenosine triphosphate (ATP), deoxynucleotides such as dAMP, dADP, and dATP, and others can be used. As the nucleic acids, deoxyribonucleic acid (DNA) or ribonucleic acid (RNA) can be used. As the nucleotides used in the present invention, from the viewpoints of taste amelioration, stability improvement, and anti-obesity effect, taste-presenting nucleotides and nucleic acids are preferred, inosinic acid, guanylic acid, deoxyribonucleic acid (DNA), and ribonucleic acid (RNA) are more preferred, and inosinic acid, DNA, and RNA are particularly preferred. The nucleotides used in the present invention may be those synthesized or those comprised in plants or animals. When the nucleotides used in the present invention are derived from plants, it is preferable that they be derived from plants other than those comprising tectorigenins.

### D. Inulins

Inulins are fructans composed of fructofuranose residues with β-D-2,1-linkages. The inulins used in the present invention may be those synthesized or those comprised in plants. When plants are used, there is no particular limitation on the types, sites, and processing methods of the plants. When the inulins used in the present invention are derived from plants, it is preferable that they be derived from plants other than those comprising tectorigenins.

### Oral Composition

In the oral composition of the present invention, there is no particular limitation on the content of the compounds of (A) to (D), but from the viewpoint of taste amelioration, stability improvement, and anti-obesity effect, it is preferably 0.00001% by weight or more, more preferably 0.00003% by weight or more, still more preferably 0.00005% by weight or more, and particularly preferably 0.0001% by weight or more with respect to the entire amount of the oral composition of the present invention. The upper limit of the content is not particularly limited, but from the viewpoint of taste amelioration, stability improvement, and anti-obesity effect, it is preferably 60% by weight or less, more preferably 30% by weight or less, still more preferably 25% by weight or less, and particularly preferably 15% by weight or less.

In the oral composition of the present invention, there is no particular limitation on the weight ratio between the tectorigenins and the compounds of (A) to (D) comprised in the oral composition, but from the viewpoint of taste amelioration, stability improvement, and anti-obesity effect, the oral composition comprises the compounds of (A) to (D) preferably in an amount of 100 parts by weight or less, more preferably in an amount of 10 parts by weight or less, still more preferably in an amount of 5 parts by weight or less, and particularly preferably in an amount of 2 parts by weight or less, with respect to 1 part by weight of the tectorigenins. In addition, from the viewpoint of taste amelioration, stability improvement, and anti-obesity effect, the oral composition comprises the compounds of (A) to (D) preferably in an amount of 0.00001 parts by weight or more, more preferably in an amount of 0.0001 parts by weight or more, still more preferably in an amount of 0.0005 parts by weight or more, and particularly preferably in an amount of 0.001 parts by weight or more, with respect to 1 part by weight of the tectorigenins. The weight of the tectorigenins in this case refers to the total amount of the weights of tectorigenin, tectoridin, and TGXG.

In the oral composition of the present invention, there is no particular limitation on the total content of the tectorigenins and the compounds of (A) to (D), but from the viewpoint of taste amelioration, stability improvement, and anti-obesity effect, it is preferably 0.001% by weight or more, more preferably 0.005% by weight or more, still more preferably 0.008% by weight or more, and particularly preferably 0.01% by weight or more with respect to the entire amount of the oral composition of the present invention. The upper limit of the content is not particularly limited, but from the viewpoint of taste amelioration, stability improvement, and anti-obesity effect, it is preferably 80% by weight or less, more preferably 40% by weight or less, still more preferably 30% by weight or less, and particularly preferably 15% by weight or less. The content of the tectorigenins in this case refers to the total amount of the contents of tectorigenin, tectoridin, and TGXG.

In the oral composition of the present invention, there is no particular limitation on the total intake amount of the tectorigenins and the compounds of (A) to (D), but from the viewpoint of taste amelioration, stability improvement, and anti-obesity effect, the total intake amount of the tectorigenins and the compounds of (A) to (D) per day is preferably 1 mg/day or more, more preferably 5 mg/day or more, still more preferably 10 mg/day or more, and particularly preferably 20 mg/day or more. The upper limit of the intake amount is not particularly limited, but from the viewpoint of taste amelioration, stability improvement, and anti-obesity effect, it is preferably 10 g/day or less, more preferably 8 g/day or less, still more preferably 5 g/day or less, and particularly preferably 3 g/day or less. The oral composition of the present invention can be accommodated in a single container so that the daily intake amount is the aforementioned intake amount, or it can be divided into and accommodated in several, for example, two to three containers as a daily dose. The intake amount of the tectorigenins in this case means the total amount of the contents of tectorigenin, tectoridin, and TGXG.

In the present invention, there is no particular limitation on the intake amount of the oral composition, but from the viewpoint of taste amelioration, stability improvement, and anti-obesity effect, the intake amount of the oral composition per day is preferably 0.01 g/day or more, more preferably 0.1 g/day or more, still more preferably 0.5 g/day or more, and particularly preferably 1 g/day or more. The upper limit of the intake amount is not particularly limited, but from the viewpoint of taste amelioration, stability improvement, and anti-obesity effect, it is preferably 100 g/day or less, more preferably 50 g/day or less, still more preferably 30 g/day or less, and particularly preferably 20 g/day or less.

The oral composition of the present invention is not particularly limited as long as it is in a form to be taken orally, and may be any of a pharmaceutical product (including a quasi-drug), so-called a health food, a food for specified health uses, or a food with functional claims, but it is preferably a food, it is more preferably a food for specified health uses or a food with functional claims, and it is particularly preferably a food with functional claims.

The oral composition of the present invention may comprise other materials normally used in addition to the tectorigenins and the compounds of (A) to (D). As the other materials, a variety of excipients, binders, brightening agents, lubricants, stabilizers, diluents, bulking agents, thickeners, emulsifiers, antioxidants, pH adjusters, colorants, flavoring agents, additives, and others can be suitably selected. The contents of materials other than the tectorigenins and the compounds of (A) to (D) can be adjusted depending on the dosage form of the present invention and other factors.

Examples of the form of the oral composition of the present invention include a tablet form, a capsule form, a powder form, a granule form, a liquid form, a rod form, a plate form, a block form, a solid form, a round form, a paste form, a cream form, a caplet form, a gel form, a chewable form, and a stick form. Among these dosage forms, from the viewpoint of manufacturability and ease of intake, a powder form, a granule form, a tablet form, and a capsule form are preferred, and a powder form, a granule form, and a tablet form are particularly preferred. Since the oral composition of the present invention has a characteristic of being easily dissolved in water, it is particularly preferred to be a powdered beverage (meaning powder or granules that are poured into water, hot water, milk, or the like and stirred for intake) or a tea bag (meaning the oral composition of the present invention in the form of powder or granules that is filled into a non-woven fabric and extracted with water or hot water for intake). The tablet, powdered beverage, and tea bag forms are also preferred from the viewpoint that they are easy to be taken at meals and other situations.

The packaging form of the oral composition of the present invention is not particularly limited, and blister packs such as PTP, strip packaging, heat seal, aluminum packaging such as aluminum pouches, film packaging using plastics, synthetic resins and the like, glass containers such as vials, plastic containers such as ampules, PET bottles, aluminum cans, steel cans, and others can be suitably selected, but from the viewpoint of storage stability of the tectorigenins, aluminum packaging is particularly preferred.

### Anti-Obesity Composition

The oral composition of the present invention can also be used as an anti-obesity composition. Ingested fat is absorbed into the body by being incorporated into particles called micelles, and the oral composition of the present invention has an action of destroying micelles. Therefore, the oral composition of the present invention exerts effects of inhibiting fat absorption and of suppressing accumulation of body fat, thus showing an excellent anti-obesity effect.

When the oral composition of the present invention is used as an anti-obesity composition, it is only required that the product be distinguishable from other products in terms of its use for obesity amelioration. For example, any product that is labeled on any of the main body, packaging, instructions, or promotional materials (advertising media) of the product according to the present invention as having a function related to obesity amelioration, such as a body fat reduction function, is included in the scope of the anti-obesity composition of the present invention. Note that the anti-obesity composition of the present invention is not limited to those in which the tectorigenins or the compounds of (A) to (D) are indicated as active ingredients on the packaging of the product or the like. For example, it may be one in which the active ingredient is not specified. In addition, even common foods are included in the scope of the anti-obesity composition of the present invention as long as they are manufactured and marketed with a suggestion of an anti-obesity application.

Specifically, for example, oral compositions indicating the following are included in the scope of the anti-obesity composition of the present invention: “Reduce body fat”, “Promote reduction in body fat”, “Suppress increase in body fat”, “Support gradual reduction in body fat”, “Reduce body fat percentage”, “Help to lower body fat percentage”, “Reduce fat on the body”, “For those with high body fat”, “For those concerned about body fat”, “Reduce BMI”, “Help to ameliorate BMI”, “Help to decrease or reduce BMI”, “Help to ameliorate high BMI value”, “For those with high BMI”, “Reduce belly fat (visceral fat)”, “For those concerned about belly fat”, “Reduce belly fat”, “For those concerned about visceral fat”, “Help to reduce visceral fat (belly fat) and BMI for those with obesity”, “Reduce waist circumference”, “Help to reduce waist circumference”, “Support gradual reduction in waist size”, “Reduce body weight”, “Help to reduce body weight”, “Promote reduction in body weight”, “Support gradual reduction in body weight”, “For those with obesity”, “For those who are overweight”, “For those concerned about body weight”, “Help those with obesity to reduce body weight and body fat”, “Increase power to decompose and consume fat”, “Further increase power to decompose and consume fat”, “Increase fat metabolism”, “Make fat consumption easier”, “Promote body fat burning during exercise”, “Reduce neutral fat”, “Suppress fat absorption”, “Support style”, “Dieting”, and others.

### EXAMPLES

Hereinafter, the present invention will be described in further detail with reference to Examples, but the present invention is not limited to these Examples and can take a variety of forms as long as they can solve the problems of the present invention.

### Organoleptic Test

The following test was performed in order to evaluate the taste of oral compositions comprising tectorigenins.

### Test Method

100 mL beverages (Examples 1 to 9 and Comparative Examples 1 to 3) were prepared so as to achieve the contents shown in Table 2. As the tectorigenins, a mixture comprising tectorigenin, tectoridin, and TGXG at a weight ratio of 4.2% by weight, 35.4% by weight, and 60.4% by weight was used. Note that the DNA used was derived from salmon milt, the RNA used was derived from yeast, and the inulins used were a commercially available synthetic product.

TABLE 2
Unit %

| | | | Comparative | | | | | | |
|---|---|---|---|---|---|---|---|---|---|
| | | | Example 1 | Example 1 | Example 2 | Example 3 | Example 4 | Example 5 | Example 6 |
| Tectorigenins | | | 0.03 | 0.03 | 0.03 | 0.03 | 0.03 | 0.03 | 0.03 |
| Amino acids | (B) | Glutamic | | 0.06 | | | | | |
| | | acid | | | | | | | |
| | | Histidine | | | 0.06 | | | | |
| | | Methionine | | | | 0.06 | | | |
| | | Phenylalanine | | | | | 0.06 | | |
| | | Tryptophan | | | | | | 0.06 | |
| | | Glycine | | | | | | | 0.06 |
| | Glutamine | | | | | | | | |
| Nucleotides | (C) | DNA | | | | | | | |
| | | RNA | | | | | | | |
| Dietary | (D) | Inulins | | | | | | | |

| | | | | | | | | |
|---|---|---|---|---|---|---|---|---|
| fiber | Indigestible | | | | | | | |
| | dextrin | | | | | | | |
| | | Remaining | Remaining | Remaining | Remaining | Remaining | Remaining | Remaining |

| Water | | amount | amount | amount | amount | amount | amount | amount |
|---|---|---|---|---|---|---|---|---|
| Total | | 100 | 100 | 100 | 100 | 100 | 100 | 100 |
| | Unit % | | | | | | | |
| | | | | Comparative | | | | Comparative |
| | | | | Example 2 | Example 7 | Example 8 | Example 9 | Example 3 |
| | Tectorigenins | | | 0.03 | 0.03 | 0.03 | 0.03 | 0.03 |
| | Amino acids | (B) | Glutamic | | | | | |
| | | | acid | | | | | |
| | | | Histidine | | | | | |
| | | | Methionine | | | | | |
| | | | Phenylalanine | | | | | |
| | | | Tryptophan | | | | | |
| | | | Glycine | | | | | |
| | | Glutamine | | 0.06 | | | | |
| | Nucleotides | (C) | DNA | | 0.03 | | | |
| | | | RNA | | | 0.03 | | |
| | Dietary | (D) | Inulins | | | | 0.6 | |

| | | | | | | |
|---|---|---|---|---|---|---|
| fiber | Indigestible | | | | | 0.6 |
| | dextrin | | | | | |
| | | Remaining | Remaining | Remaining | Remaining | Remaining |

| | | | | | |
|---|---|---|---|---|---|
| Water | amount | amount | amount | amount | amount |
| Total | 100 | 100 | 100 | 100 | 100 |

Five subjects were given the beverages of Examples and Comparative Examples to carry out evaluation for the taste such as deliciousness. For the evaluation, each of Examples and Comparative Examples were graded according to the evaluation criteria described below, using Comparative Example 1 as the standard (0 point), and the average score was calculated.

| | |
|---|---|
| 3 | very good |
| 2 | good |
| 1 | rather good |
| 0 | no change |
| −1 | rather bad |
| −2 | bad |
| −3 | very bad |

### Test Results

The results of the organoleptic test are shown in Table 3.

TABLE 3

| | Comparative | | | | | | |
|---|---|---|---|---|---|---|---|
| Item | Example 1 | Example 1 | Example 2 | Example 3 | Example 4 | Example 5 | Example 6 |
| Deliciousness | 0 | 1.6 | 1.6 | 0.2 | 1.4 | 1.6 | 1.8 |
| Sweetness | 0 | 1 | 1.6 | 0.8 | 1.6 | 1.6 | 1.6 |
| Umami taste | 0 | 2 | 1.6 | 0.8 | 1.6 | 1.6 | 1.8 |
| Richness | 0 | 1.6 | 2 | 1 | 1.6 | 1.4 | 1.6 |
| of taste | | | | | | | |
| Aftertaste | 0 | 1 | 1 | 0.2 | 1.4 | 1.4 | 1.4 |
| Richness | 0 | 1.6 | 1.8 | 0.8 | 1.4 | 1.4 | 1.6 |
| (hearty taste) | | | | | | | |

| | Comparative | | | | Comparative |
|---|---|---|---|---|---|
| Item | Example 2 | Example 7 | Example 8 | Example 9 | Example 3 |
| Deliciousness | −0.8 | 1.6 | 2.2 | 2.4 | 0 |
| Sweetness | −0.2 | 1.6 | 1.6 | 2 | −0.4 |
| Umami taste | 0 | 1.8 | 1.8 | 2.2 | 0 |
| Richness | −0.2 | 1.6 | 2.2 | 2.2 | 0.4 |
| of taste | | | | | |
| Aftertaste | −1 | 2 | 2.2 | 1.6 | −1 |
| Richness | −0.6 | 1.6 | 1.8 | 2 | 0 |
| (hearty taste) | | | | | |

The beverages (Examples 1 to 9) comprising the specific compounds described in (B) to (D) in addition to the tectorigenins showed ameliorated taste compared to the beverage (Comparative Example 1) comprising only the tectorigenins. On the other hand, the beverage (Comparative Example 2) comprising glutamine, which is an amino acid, and the beverage (Comparative Example 3) comprising indigestible dextrin, which is a dietary fiber, did not show amelioration in taste. Note that all of the oral compositions of the present invention had high solubility in water.

### Stability Test

In order to evaluate the stability of the tectorigenins in solutions, the following test was performed.

### Test Method

Diluted solutions of tectorigenins with anthocyanins, quercetins, and catechins were created, and by fractionating the diluted solutions, 50% aqueous ethanol solutions comprising the tectorigenins with any of the anthocyanins, quercetins, or catechins were prepared so as to achieve the contents shown in Table 4. As the raw material tectorigenins, a mixture comprising tectorigenin, tectoridin, and TGXG at a weight ratio of 4.2% by weight, 35.4% by weight, and 60.4% by weight was used. The anthocyanins used were derived from elderberry, and the quercetins used were quercetin dihydrate.

TABLE 4

| | | | Comparative | | | Comparative |
|---|---|---|---|---|---|---|
| | | | Example 4 | Example 10 | Example 11 | Example 5 |
| Tectorigenins | | | 0.075 | 0.075 | 0.075 | 0.075 |
| Polyphenol | (A) | Anthocyanins | | 0.075 | | |
| | | Quercetins | | | 0.075 | |

| | |
|---|---|
| Catechins | 0.075 |

Unit mg/mL

10 mL of each prepared solution was fractionated into each of two polypropylene centrifuge tubes (for 15 mL), stored for 24 hours under the following conditions (a) and (b), and the amount of the tectorigenins in each solution after 24 hours was measured. Based on the measured values obtained, the reduction rate of the tectorigenins was calculated using the following calculation expression (Formula 1). The results are shown in Table 5.
(a) Dark room, 5 to 7° C. (refrigerated storage)
(b) Near window (exposed to sunlight), 20° C. (stored at room temperature)

$$Reduction rate ( \% ) = 100 \times ( \frac{( b )}{( a )} - 1 ) \qquad [ Formula 1 ]$$

TABLE 5

| | Comparative | | | Comparative |
|---|---|---|---|---|
| | Example 4 | Example 10 | Example 11 | Example 5 |
| Reduction | −5% | 0% | 0% | −4% |
| rate of | | | | |
| tectorigenins | | | | |

The solutions in which the anthocyanins or quercetins, which are the compounds of (A) in the present invention, were added to the tectorigenins (Example 10 and Example 11) showed suppressed reduction in the tectorigenins compared to the solution (Comparative Example 4) in which no compounds were added. On the other hand, the reduction rate of the solution (Comparative Example 5) to which catechins, a type of polyphenol, were added was similar to that of Comparative Example 4.

### Anti-Obesity Test (Micelle Structure Breakdown Test)

In order to evaluate the anti-obesity effect, the following test was performed.

### Test Method

### Preparation of Control Solution and Test Substances

A 0.2 M Tris-HCl buffer (pH 7.0) was prepared and used as the control solution. In addition, each compound was added to the 0.2 M Tris-HCl buffer (pH 7.0) so as to achieve the concentrations described in Tables 6 to 9, thereby preparing the test substances. As the tectorigenins, a mixture comprising tectorigenin, tectoridin, and TGXG at a weight ratio of 4.2% by weight, 35.4% by weight, and 60.4% by weight was used. The anthocyanins used were derived from elderberry, the quercetins used were quercetin dihydrate, the RNA used was derived from yeast, and the inulins used were a commercially available synthetic product.

TABLE 6

| | | | | Comparative | Comparative | Comparative | Comparative |
|---|---|---|---|---|---|---|---|
| | | | | Example 6 | Example 7 | Example 8 | Example 9 |
| Concentration | Tectorigenins | | | 12.5 | | | |
| in test | Polyphenol | (A) | Anthocyanins | | 12.5 | | |
| substances | | | Quercetins | | | 12.5 | |
| (mg/mL) | | | | | | | 12.5 |
| | Total | | | 12.5 | 12.5 | 12.5 | 12.5 |
| Breakdown rate (%) | | | | 6.1 | 4.4 | 7.9 | 3.2 |
| | | | | | Comparative | | |
| | | | | | Example 10 | Example 12 | Example 13 |
| | Concentration | Tectorigenins | | | 6.25 | 6.25 | 6.25 |
| | in test | Polyphenol | (A) | Anthocyanins | | 6.25 | |
| | substances | | | Quercetins | | | 6.25 |

| | |
|---|---|
| (mg/mL) | 6.25 |

| | | | |
|---|---|---|---|
| Total | 12.5 | 12.5 | 12.5 |

| | | | |
|---|---|---|---|
| Breakdown rate (%) | 1.8 | 31.5 | 61.4 |

TABLE 7

| | | | | Comparative | Comparative | Comparative | Comparative | Comparative |
|---|---|---|---|---|---|---|---|---|
| | | | | Example 11 | Example 12 | Example 13 | Example 14 | Example 15 |
| Concentration | Tectorigenins | | | 50 | | | | |
| in test | Amino acids | (B) | Glutamic | | 50 | | | |
| substances | | | acid | | | | | |
| (mg/mL) | | | Histidine | | | 50 | | |
| | | | Methionine | | | | 50 | |
| | | | Phenylalanine | | | | | 50 |
| | | | Tryptophan | | | | | |
| | | | [symbol] | | | | | |
| | | [symbol] | | | | | | |
| | Polyphenyl | (A) | Anthocyanins | | | | | |
| | Nucelotide | (C) | [symbol] acid | | | | | |
| | Total | | | 50 | 50 | 50 | 50 | 50 |
| Breakdown rate (%) | | | | 4.6 | 21.7 | 1.8 | 24.4 | 19.9 |
| | | | | Comparative | Comparative | Comparative | Comparative | Comparative |
| | | | | Example 16 | Example 17 | Example 18 | Example 19 | Example 20 |
| Concentration | Tectorigenins | | | | | | | |
| in test | Amino acids | (B) | Glutamic | | | | | |
| substances | | | acid | | | | | |
| (mg/mL) | | | Histidine | | | | | |
| | | | Methionine | | | | | |
| | | | Phenylalanine | | | | | |
| | | | Tryptophan | 50 | | | | |
| | | | [symbol] | | 50 | | | |
| | | [symbol] | | | | 50 | | |
| | Polyphenyl | (A) | Anthocyanins | | | | 50 | |
| | Nucelotide | (C) | [symbol] acid | | | | | 50 |
| | Total | | | 50 | 50 | 50 | 50 | 50 |
| Breakdown rate (%) | | | | 1 | 1.8 | 15.2 | 19.8 | 6.7 |
| | | | | Comparative | | | | |
| | | | | Example 21 | Example 14 | Example 15 | Example 16 | Example 17 |
| Concentration | Tectorigenins | | | 25 | 25 | 25 | 25 | 25 |
| in test | Amino acids | (B) | Glutamic | | 25 | | | |
| substances | | | acid | | | | | |
| (mg/mL) | | | Histidine | | | 25 | | |
| | | | Methionine | | | | 25 | |
| | | | Phenylalanine | | | | | 25 |
| | | | Tryptophan | | | | | |
| | | | [symbol] | | | | | |
| | | [symbol] | | 25 | | | | |
| | Polyphenyl | (A) | Anthocyanins | | | | | |
| | Nucelotide | (C) | [symbol] acid | | | | | |
| | Total | | | 50 | 50 | 50 | 50 | 50 |
| Breakdown rate (%) | | | | 11 | [symbol] | [symbol] | 48.5 | 43.3 |
| | | | | | Example 18 | Example 19 | Example 20 | Example 21 |
| | Concentration | Tectorigenins | | | 25 | 25 | 25 | 25 |
| | in test | Amino acids | (B) | Glutamic | | | | |
| | substances | | | acid | | | | |
| | (mg/mL) | | | Histidine | | | | |
| | | | | Methionine | | | | |
| | | | | Phenylalanine | | | | |
| | | | | Tryptophan | 25 | | | |
| | | | | [symbol] | | 25 | 12.5 | 12.5 |

| | [symbol] | | | |
|---|---|---|---|---|
| Polyphenyl | (A) | Anthocyanins | 12.5 | |
| Nucelotide | (C) | [symbol] acid | | 12.5 |

| | | | | |
|---|---|---|---|---|
| Total | 50 | 50 | 50 | 50 |

| | | | | |
|---|---|---|---|---|
| Breakdown rate (%) | 42.3 | 21.8 | 69.5 | 66.3 |

[symbol]

indicates data missing or illegible when filed

TABLE 8

| | | | | Comparative | Comparative | Comparative | Comparative |
|---|---|---|---|---|---|---|---|
| | | | | Example 22 | Example 23 | Example 24 | Example 25 |
| Concentration | Tectorigenins | | | 50 | | | |
| in test | Nucleotides | (C) | RNA | | 50 | | |
| substances | | | Inosinic | | | 50 | |
| (mg/mL) | | | acid | | | | |
| | Polyphenyl | (A) | Anthocyanins | | | | 50 |
| | Total | | | 50 | 50 | 50 | 50 |
| Breakdown rate (%) | | | | 4.6 | 4.3 | 6.7 | 10.8 |
| | | | | | Example 22 | Example 23 | Example 24 |
| | Concentration | Tectorigenins | | | 25 | 25 | 25 |
| | in test | Nucleotides | (C) | RNA | 25 | | |
| | substances | | | Inosinic | | 25 | 12.5 |
| | (mg/mL) | | | acid | | | |
| | | Polyphenyl | (A) | Anthocyanins | | | 12.5 |

| | | | |
|---|---|---|---|
| Total | 50 | 50 | 50 |

| | | | |
|---|---|---|---|
| Breakdown rate (%) | 38.4 | 29.8 | 69.1 |

TABLE 9

| | | | | Comparative | Comparative | Comparative | Comparative | |
|---|---|---|---|---|---|---|---|---|
| | | | | Example 26 | Example 27 | Example 28 | Example 29 | Example 25 |
| Concentration | Tectorigenins | | | 50 | | | 25 | 25 |
| in test | Dietary | (D) | Inulins | | 50 | | | 25 |
| substances | fiber | | | | | | | |

| | | | |
|---|---|---|---|
| (mg/mL) | Indigestible | 50 | 25 |
| | dextrin | | |

| | | | | | |
|---|---|---|---|---|---|
| Total | 50 | 50 | 50 | 50 | 50 |

| | | | | | |
|---|---|---|---|---|---|
| Breakdown rate (%) | 4.6 | 10.5 | 16.9 | 20.5 | 48.2 |

### Preparation of Micelle-Formed Emulsion

After mixing 2 mL of olive oil (manufactured by Nacalai Tesque, Inc.), 23 mL of ultrapure water, and sodium cholate (manufactured by Nacalai Tesque, Inc.), the resultant was treated with ultrasound (VP-5S, manufactured by Taitec Corporation) for 6 minutes to prepare an emulsion with an absorbance of 0.5 or more at a wavelength of 500 nm. The sodium cholate concentration of the emulsion was adjusted to 0.2%. Note that it is generally known that the absorbance at 500 nm is an index of the amount of micelles formed (see, for example, “Journal of Japanese Society of Nutrition and Food Science, Vol. 60, No. 2 (2007), pp. 105-110”).

### Measurement of Micelle Breakdown Rate

The prepared emulsion was mixed with the control solution or test substances at a ratio of 1:1, adjusted so that the final concentration of sodium cholate was 0.1%, and shaken (Bioshaker, BR-43F, manufactured by Taitec Corporation) at 37° C. for 120 minutes. The shaking rate was set to 80 shakes/min. Before and after the shaking, the mixed solution was diluted by 100 times with a 0.1% sodium dodecyl sulfate (SDS, manufactured by FUJIFILM Wako Pure Chemical Corporation) solution, and the absorbance at 500 nm was measured. Based on the measured absorbance, the micelle breakdown rate (%) was calculated using the following calculation expression. The measured micelle breakdown rates are shown in Tables 6 to 9.

$$Micelle breakdown rate ( \% ) = \frac{\begin{matrix}Test {substance}^{Before shaking} - \\ Test {substance}^{After shaking}\end{matrix}}{{Control}^{Test} - {Control}^{Blank}} \times 100 \qquad [ Expression 2 ]$$

Test substance^{Before shaking}=Absorbance (500 nm) of mixed solution of emulsion and test substance before shaking
Test substance^{After shaking}=Absorbance (500 nm) of mixed solution of emulsion and test substance after shaking
Control^{Test}=Absorbance (500 nm) of mixed solution of emulsion and control solution after shaking Control^{Blank}=Absorbance (500 nm) of control solution alone after shaking

### Test Results

Examples 12 to 25, comprising the tectorigenins and the compounds of (A) to (D), had significantly higher micelle breakdown rates compared to those of the test substances comprising only the tectorigenins, or Comparative Examples 6 to 9, 11 to 20, and 22 to 28, comprising only the compounds of (A) to (D). Improvement in the micelle breakdown rate by combining the tectorigenins with the compounds of (A) to (D) over those alone is an effect of the present invention. Also, Examples 20, 21, and 24, comprising two types of the compounds of (A) to (D) in addition to the tectorigenins, showed an even more significant increase in the proportion of broken micelles compared to Examples 19 and 23, comprising only one type of the compounds of (A) to (D).

Since fat absorption is suppressed when the micelle structure is broken down in vivo, the breakdown of the micelle structure is an index of the anti-obesity effect. The micelle breakdown rate is significantly increased by comprising the compounds of (A) to (D) in addition to the tectorigenins, thus making the oral composition of the present invention useful as an anti-obesity composition or a body fat-reducing composition.

### PRODUCTION EXAMPLES

Hereinafter, Production Examples of the present invention will be shown.

### Production Examples 1 to 18: Powdered Beverages in Granular Form

As shown in Table 10, after mixing each raw material, fluid bed granulation was carried out using a granulator to produce powdered beverages in the form of granules as described in Production Examples 1 to 18. The powdered beverages produced were filled into aluminum pouches so that each pouch contained 3 g. By filling the powdered beverages into aluminum pouches, the tectorigenins are stored in a stable manner. The powdered beverages described in Production Examples 1 to 18 are only required to be taken once a day at a dose of 3 g per intake. The powdered beverages of Production Examples 1 to 18 are easily dispersed in water or hot water and thus can be taken with a little stirring. In addition, all of the powdered beverages of Production Examples 1 to 18 are excellent in palatability and effective in anti-obesity. In particular, powdered beverages comprising two or more types of the compounds of (A) to (D) in addition to the tectorigenins exert an excellent anti-obesity effect.

TABLE 10

| | | Production Examples | | | | | | | | | |
|---|---|---|---|---|---|---|---|---|---|---|---|
| Powdered beverages | | 1 | 2 | 3 | 4 | 5 | 6 | 7 | 8 | 9 | 10 |
| Tectorigenins | Tectorigenin | 2.0 | | | 0.2 | 0.2 | 0.2 | 2.0 | | | 0.2 |
| | Tectoridin | | 2.0 | | 0.3 | 0.3 | 0.3 | | 2.0 | | 0.3 |
| | TGXG | | | 2.0 | 1.0 | 1.0 | 1.0 | | | 2.0 | 1.0 |
| (A) | Anthocyanins | 1.0 | | | | | | | | | |
| | Quercetins | | 1.0 | | | | | | | | |
| (B) | Glutamic acid | | | 1.0 | | | | | | | |
| | Histidine | | | | 1.0 | | | | | | |
| | Methionine | | | | | 1.0 | | | | | |
| | Phenylalanine | | | | | | 1.0 | | | | |
| | Tryptophan | | | | | | | 1.0 | | | |
| | Cysteine | | | | | | | | 1.0 | | |
| | Glycine | | | | | | | | | 1.0 | |
| (C) | DNA (derived | | | | | | | | | | 1.0 |
| | from salmon milt) | | | | | | | | | | |
| | RNA (derived | | | | | | | | | | |
| | from yeast) | | | | | | | | | | |
| | [symbol] acid | | | | | | | | | | |
| | [symbol] acid | | | | | | | | | | |
| (D) | Inulins | | | | | | | | | | |
| Others | Reduced palatinose | 77.0 | 77.0 | 77.0 | 77.5 | 77.5 | 77.5 | 77.0 | 77.0 | 77.0 | 77.5 |
| | Sucrose | 20.0 | 20.0 | 20.0 | 20.0 | 20.0 | 20.0 | 20.0 | 20.0 | 20.0 | 20.0 |

| | | | | | | | | | | |
|---|---|---|---|---|---|---|---|---|---|---|
| Total | 100 | 100 | 100 | 100 | 100 | 100 | 100 | 100 | 100 | 100 |

| | | Production Examples | | | | | | | |
|---|---|---|---|---|---|---|---|---|---|
| Powdered beverages | | 11 | 12 | 13 | 14 | 15 | 16 | 17 | 18 |
| Tectorigenins | Tectorigenin | 0.2 | 0.2 | 0.2 | 0.2 | 0.2 | 0.2 | 0.2 | 0.2 |
| | Tectoridin | 0.3 | 0.3 | 0.3 | 0.3 | 0.3 | 0.3 | 0.3 | 0.3 |
| | TGXG | 1.0 | 1.0 | 1.0 | 1.0 | 1.0 | 1.0 | | 0.1 |
| (A) | Anthocyanins | | | | | 0.5 | 0.5 | | 0.1 |
| | Quercetins | | | | | 0.5 | 0.5 | | 0.1 |
| (B) | Glutamic acid | | | | | 0.1 | | 0.1 | 0.1 |
| | Histidine | | | | | 0.1 | | 0.1 | 0.1 |
| | Methionine | | | | | 0.1 | | 0.1 | 0.1 |
| | Phenylalanine | | | | | 0.1 | | 0.1 | 0.1 |
| | Tryptophan | | | | | 0.1 | | 0.1 | 0.1 |
| | Cysteine | | | | | 0.1 | | 0.1 | 0.1 |
| | Glycine | | | | | 0.1 | | 0.1 | 0.1 |
| (C) | DNA (derived | | | | | | 0.2 | 0.1 | 0.1 |
| | from salmon milt) | | | | | | | | |
| | RNA (derived | 1.0 | | | | | 0.2 | 0.1 | 0.1 |
| | from yeast) | | | | | | | | |
| | [symbol] acid | | 1.0 | | | | 0.3 | 0.1 | 0.1 |
| | [symbol] acid | | | 1.0 | | | 0.3 | 0.1 | 0.1 |
| (D) | Inulins | | | | 1.0 | | | | 0.1 |
| Others | Reduced palatinose | 77.5 | 77.5 | 77.5 | 77.5 | 76.8 | 76.5 | 78.4 | 78.0 |
| | Sucrose | 20.0 | 20.0 | 20.0 | 20.0 | 20.0 | 20.0 | 20.0 | 20.0 |

| | | | | | | | | |
|---|---|---|---|---|---|---|---|---|
| Total | 100 | 100 | 1002 | 100 | 100 | 100 | 100 | 100 |

[symbol]

indicates data missing or illegible when filed

### Production Examples 19 to 36: Tablet

As shown in Table 11, after mixing each raw material, tableting was carried out using a rotary tableting machine to produce tablets of Production Examples 19 to 36. The tablets were produced with a tablet diameter of 8 mmφ, a tablet thickness of 4.5 mm, a weight of 300 mg, and a hardness of 5 kgf or more. For the tablets described in Production Examples 19 to 36, 5 to 6 tablets are only required to be taken per day, and can be taken together with water or the like. By packaging the tablets with aluminum pouches, the tectorigenins are stored in a stable manner. In addition, all of the tablets of Production Examples 19 to 36 are excellent in palatability and effective in anti-obesity. In particular, tablets comprising two or more types of the compounds of (A) to (D) in addition to the tectorigenins exert an excellent anti-obesity effect.

TABLE 11

| | | Production Examples | | | | | | | | |
|---|---|---|---|---|---|---|---|---|---|---|
| Tablet | | 19 | 20 | 21 | 22 | 23 | 24 | 25 | 26 | 27 |
| Tectorigenins | Tectorigenin | 1.0 | 1.0 | 1.0 | 1.0 | 1.0 | 3.0 | | | 1.0 |
| | Tectoridin | 1.0 | 1.0 | 1.0 | 1.0 | 1.0 | | 3.0 | | 1.0 |
| | TGXG | 1.0 | 1.0 | 1.0 | 1.0 | 1.0 | | | 3.0 | 1.0 |
| (A) | Anthocyanins | 1.0 | | | | | | | | |
| | Quercetins | | 1.0 | | | | | | | |
| (B) | Glutamic acid | | | 1.0 | | | | | | |
| | Histidine | | | | 1.0 | | | | | |
| | Methionine | | | | | 1.0 | | | | |
| | Phenylalanine | | | | | | 1.0 | | | |
| | Tryptophan | | | | | | | 1.0 | | |
| | Cysteine | | | | | | | | 1.0 | |
| | Glycine | | | | | | | | | 1.0 |
| (C) | DNA (derived | | | | | | | | | |
| | from salmon milt) | | | | | | | | | |
| | RNA (derived | | | | | | | | | |
| | from yeast) | | | | | | | | | |
| | Inosinic acid | | | | | | | | | |
| | [symbol] acid | | | | | | | | | |
| (D) | Inulins | | | | | | | | | |
| Others | Maltose | 30.0 | 30.0 | 30.0 | 30.0 | 30.0 | 30.0 | 30.0 | 30.0 | 30.0 |
| | Reduced maltose | 33.0 | 33.0 | 33.0 | 33.0 | 33.0 | 33.0 | 33.0 | 33.0 | 33.0 |
| | Reduced palatinose | 30.0 | 30.0 | 30.0 | 30.0 | 30.0 | 30.0 | 30.0 | 30.0 | 30.0 |
| | Silicon dioxide | 1.0 | 1.0 | 1.0 | 1.0 | 1.0 | 1.0 | 1.0 | 1.0 | 1.0 |
| | Calcium stearate | 2.0 | 2.0 | 2.0 | 2.0 | 2.0 | 2.0 | 2.0 | 2.0 | 2.0 |
| Total | | 100 | 100 | 100 | 100 | 100 | 100 | 100 | 100 | 100 |
| | | Production Examples | | | | | | | | |
| Tablet | | 28 | 29 | 30 | 31 | 32 | 33 | 34 | 35 | 36 |
| Tectorigenins | Tectorigenin | 1.0 | 1.0 | 3.0 | | | 1.0 | 1.0 | | 1.0 |
| | Tectoridin | 1.0 | 1.0 | | 3.0 | | 1.0 | 1.0 | | 1.0 |
| | TGXG | 1.0 | 1.0 | | | 3.0 | 1.0 | 1.0 | | 0.1 |
| (A) | Anthocyanins | | | | | | 0.5 | 0.5 | | 0.1 |
| | Quercetins | | | | | | 0.5 | 0.5 | | 0.1 |
| (B) | Glutamic acid | | | | | | 0.1 | | 0.1 | 0.1 |
| | Histidine | | | | | | 0.1 | | 0.1 | 0.1 |
| | Methionine | | | | | | 0.1 | | 0.1 | 0.1 |
| | Phenylalanine | | | | | | 0.1 | | 0.1 | 0.1 |
| | Tryptophan | | | | | | 0.1 | | 0.1 | 0.1 |
| | Cysteine | | | | | | 0.1 | | 0.1 | 0.1 |
| | Glycine | | | | | | 0.1 | | 0.1 | 0.1 |
| (C) | DNA (derived | 1.0 | | | | | | 0.2 | 0.1 | 0.1 |
| | from salmon milt) | | | | | | | | | |
| | RNA (derived | | 1.0 | | | | | 0.2 | 0.1 | 0.1 |
| | from yeast) | | | | | | | | | |
| | Inosinic acid | | | 1.0 | | | | 0.3 | 0.1 | 0.1 |
| | [symbol] acid | | | | 1.0 | | | 0.3 | 0.1 | 0.1 |
| (D) | Inulins | | | | | 1.0 | | | | 0.1 |
| Others | Maltose | 30.0 | 30.0 | 30.0 | 30.0 | 30.0 | 30.0 | 30.0 | 30.0 | 30.0 |
| | Reduced maltose | 33.0 | 33.0 | 33.0 | 33.0 | 33.0 | 32.3 | 32.0 | 33.9 | 33.5 |
| | Reduced palatinose | 30.0 | 30.0 | 30.0 | 30.0 | 30.0 | 30.0 | 30.0 | 30.0 | 30.0 |
| | Silicon dioxide | 1.0 | 1.0 | 1.0 | 1.0 | 1.0 | 1.0 | 1.0 | 1.0 | 1.0 |
| | Calcium stearate | 2.0 | 2.0 | 2.0 | 2.0 | 2.0 | 2.0 | 2.0 | 2.0 | 2.0 |

| | | | | | | | | | |
|---|---|---|---|---|---|---|---|---|---|
| Total | 100 | 100 | 100 | 100 | 100 | 100 | 100 | 100 | 100 |

[symbol]

indicates data missing or illegible when filed

### Production Examples 37 to 54: Tea Bag

As shown in Table 12, each raw material was mixed and filled into a non-woven fabric so that each bag contained 2 g, thereby producing tea bags. By packaging the tea bags with aluminum pouches, the tectorigenins are stored in a stable manner. The tea bags described in Production Examples 37 to 54 can be taken by, for example, extraction with 150 mL of hot water for 1 minute. In addition, all of the tea bags of Production Examples 37 to 54 are excellent in palatability and effective in anti-obesity. In particular, tea bags comprising two or more types of the compounds of (A) to (D) in addition to the tectorigenins exert an excellent anti-obesity effect.

TABLE 12

| | | Production Examples | | | | | | | | |
|---|---|---|---|---|---|---|---|---|---|---|
| Tea bag | | 37 | 38 | 39 | 40 | 41 | 42 | 43 | 44 | 45 |
| Tectorigenins | Tectorigenin | 2.0 | | | 0.1 | 0.1 | 0.1 | 0.1 | 2.0 | |
| | Tectoridin | | 2.0 | | 0.4 | 0.4 | 0.4 | 0.4 | | 2.0 |
| | TGXG | | | 2.0 | 1.5 | 1.5 | 1.5 | 1.5 | | |
| (A) | Anthocyanins | 1.0 | | | | | | | | |
| | Quercetins | | 1.0 | | | | | | | |
| (B) | Glutamic acid | | | 1.0 | | | | | | |
| | Histidine | | | | 1.0 | | | | | |
| | Methionine | | | | | 1.0 | | | | |
| | Phenylalanine | | | | | | 1.0 | | | |
| | Tryptophan | | | | | | | 1.0 | | |
| | Cysteine | | | | | | | | 1.0 | |
| | Glycine | | | | | | | | | 1.0 |
| (C) | DNA (derived | | | | | | | | | |
| | from salmon milt) | | | | | | | | | |
| | RNA (derived | | | | | | | | | |
| | from yeast) | | | | | | | | | |
| | Inosinic acid | | | | | | | | | |
| | [symbol] acid | | | | | | | | | |
| (D) | [symbol] | | | | | | | | | |
| Others | Green tea dried powder | 97.0 | 97.0 | 97.0 | 97.0 | 97.0 | 97.0 | 97.0 | 97.0 | 97.0 |
| | Black tea dried powder | | | | | | | | | |
| Total | | 100 | 100 | 100 | 100 | 100 | 100 | 100 | 100 | 100 |
| | | Production Examples | | | | | | | | |
| Tea bag | | 46 | 47 | 48 | 49 | 50 | 51 | 52 | 53 | 54 |
| Tectorigenins | Tectorigenin | | 0.1 | 0.1 | 0.1 | 2.0 | | | 0.1 | 0.1 |
| | Tectoridin | | 0.4 | 0.4 | 0.4 | | 2.0 | | 0.4 | 0.4 |
| | TGXG | 2.0 | 1.5 | 1.5 | 1.5 | | | 2.0 | 1.5 | 1.5 |
| (A) | Anthocyanins | | | | | | 1.0 | 1.0 | | 0.1 |
| | Quercetins | | | | | | 0.5 | 0.5 | | 0.1 |
| (B) | Glutamic acid | | | | | | 0.5 | 0.5 | | 0.1 |
| | Histidine | | | | | | 0.1 | | 0.1 | 0.1 |
| | Methionine | | | | | | 0.1 | | 0.1 | 0.1 |
| | Phenylalanine | | | | | | 0.1 | | 0.1 | 0.1 |
| | Tryptophan | | | | | | 0.1 | | 0.1 | 0.1 |
| | Cysteine | | | | | | 0.1 | | 0.1 | 0.1 |
| | Glycine | | | | | | 0.1 | | 0.1 | 0.1 |
| (C) | DNA (derived | 1.0 | | | | | 0.1 | | 0.1 | 0.1 |
| | from salmon milt) | | | | | | | | | |
| | RNA (derived | | 1.0 | | | | | 0.2 | 0.1 | 0.1 |
| | from yeast) | | | | | | | | | |
| | Inosinic acid | | | 1.0 | | | | 0.2 | 0.1 | 0.1 |
| | [symbol] acid | | | | 1.0 | | | 0.3 | 0.1 | 0.1 |
| (D) | [symbol] | | | | | 1.0 | | 0.3 | 0.1 | 0.1 |
| Others | Green tea dried powder | 97.0 | | | | | | | | 0.1 |
| | Black tea dried powder | | 97.0 | 97.0 | 97.0 | 97.0 | 95.3 | 95.0 | 96.9 | 96.5 |

| | | | | | | | | | |
|---|---|---|---|---|---|---|---|---|---|
| Total | 100 | 100 | 100 | 100 | 100 | 100 | 100 | 100 | 100 |

[symbol]

indicates data missing or illegible when filed

### INDUSTRIAL APPLICABILITY

The oral composition of the present invention exerts excellent palatability or storage stability of tectorigenins and is thus of high industrial usefulness.

1. A method for suppressing obesity or for reducing body fat, comprising administering to a human an oral composition comprising tectorigenins, and one or more compounds selected from the group consisting of (A) to (D):
(A) one or more polyphenols selected from anthocyanins and quercetins;
(B) one or more amino acids selected from glutamic acid, histidine, methionine, phenylalanine, tryptophan, cysteine, and glycine;
(C) nucleotides; and
(D) inulins.

2. The method according to claim 1, wherein the oral composition is in a powder form, a granule form, a tablet form, or a capsule form.

3. The method according to claim 1, wherein the oral composition is a tea bag.
